(19) United States
(12) Patent Application Publication
(10) Pub. No.: US 2026/0258399 A1
(43) Pub. Date: Sep. 3, 2026

(54) SMALL MOLECULE BARCODING TO SCREEN WHOLE PREMADE, SYNTHESIZED LIBRARIES OF CHEMICAL ENTITIES

(71) Applicant: INTERPLAY BIO, Ann Arbor, MI (US)

(72) Inventor: Pavan Kommareddi, Ypsilanti, MI (US)

(73) Assignee: INTERPLAY BIO, Ann Arbor, MI (US)

(21) Appl. No.: 19/068,478

(22) Filed: Mar. 3, 2025

(51) Int. Cl. C12N 15/10

(52) U.S. Cl. CPC C12N 15/1065; C12N 15/1037

(57) ABSTRACT

The disclosed method directly conjugates one of a DNA-based barcode, a peptide-based barcode, or a fluorescent bead based-barcode onto small molecule whereby existing libraries of chemical entities may be modified through the barcoding of small molecules. The process utilizes a linker to link any molecule with an aminated molecule. A barcoded small molecule library is created through the combination of diazirine used to attach small molecules to DNA, peptide, and fluorescent microspheres followed by radiation. The exposed molecules are then photo-linked resulting in immobilization to the barcodes followed by complementary sequencing. This tri-modal barcoding system integrates with phage display and imaging-based analyzers to accelerate drug discovery by identifying novel protein-ligand interactions efficiently. The resulting barcoded library may be bound with biotin through the use of an inert substrate followed by incubation and then washing to remove unbound material. The small molecules are subjected to PCR to identify DNA encoded molecules.

### DESCRIPTION OF THE INVENTION

### Technical Field

The disclosed invention relates generally to expanding the utilization of libraries of chemical entities. More particularly, the disclosed invention relates to a system and method for barcoding small molecules for use in screening whole premade, synthesized, small molecule libraries of chemical entities through the use of DNA-based, peptide-based, and fluorescent micro bead-based barcoding. The presence of barcoding assists researchers in the high throughput screening of small molecule-protein interactions to accelerate known drug discovery platforms for the identification of previously unknown protein-small molecule interactions that will lead to the development of first-in-class drugs for a variety of targets, including currently undruggable targets.

### BACKGROUND OF THE INVENTION

Development of new drugs is known to be a very time-consuming and expensive process. Today, it is common for the development of a new drug, working from the very first step, to take between about twelve and fifteen years and cost between about two and three billion dollars. There are many false starts associated with new drug development and, as a consequence, the time for research and associated costs are constantly increasing. This slow development is the main reason that of the more than 10,000 known diseases, only a very small proportion, about 500, have treatments.

As a consequence, an increasing amount of attention is being given to a more efficient and cost-effective approach to drug discovery compared to traditional high throughput screening processes. During drug development, it is common for the drug researchers to target specific diseases. Little consideration is understandably given at the time of development to alternative or potential applications of the drug to other disease states, particularly those that are not related to the disease initially being targeted. Compounding the problem is that new drugs undergo extensive regulatory review coupled with an ever-expanding approval period. Despite strong efforts, it is clear that investment of both time and resources into drug discovery fall short of realizing the true potential of drug discovery methodologies.

Efforts have been made to find efficient and cost-effective approaches to discover new drugs even for targets previously believed to be undruggable. It is believed that it is possible to reduce drug development time by between about five and seven times and reducing development costs by between about six and ten times when compared with standard de novo development techniques. However, the strategy of drug development is slowed by several factors, including the need for a disease screening model, the need for individual processes for every disease model, and the fact that this multi-step process requires both disease screening and subsequent protein target identification. Today's drug development protocols are also limited by the phenotype disease model. As a result, known approaches to drug development are faced with lost time, a situation often made worse by the limitations of the initial treatment hypothesis.

Methodologies used for the development of new drugs today limit the speed of drug development research as they typically involve the time-consuming screening of protein-small molecule binding interactions. While providing useful advances in alternative or additional drug application discovery, known screening methods are burdened by overly complex protocols. According to the most common approach, a bacteriophage display library is first established after which the protein-small molecule interactions are identified and evaluated once immobilization of the small molecule takes place. However, this technique relies on altering the chemistry of each molecule under investigation, thus resulting in a cumbersome and slow process.

Such efforts have included various techniques for identifying the possible repurposing of known drugs. Some of these approaches involve the use of various platforms used for analyzing protein-small molecule interactions. Examples of such research platforms include the above-referenced SUMOTAC (SUMO Targeting Chimera), DUBTAC (Deubiquitinase Targeting Chimera), and PROTAC (Proteolysis-Targeting Chimera) platforms. In particular, the PROTAC platform provides a useful study tool which, in particular, provides small molecule-induced protein degradation technology. In particular, the PROTAC platform uses bifunctional small molecules to induce ubiquitination of target proteins and utilizes intracellular proteasomes for chemical knockdown. Molecular glues are small molecules that are capable of facilitating protein-protein interactions. These interactions are achieved by binding to two proteins and bringing them together. Accordingly, molecular glues function as a bridge between the proteins. As a result, the proteins are capable of interacting in an abnormal way.

Efforts have been made to overcome the challenges faced by known drug development protocols and have been either been patented, such as that found in U.S. Pat. No. 6,514,295B1 titled “Precision Fluorescently Dyed Particles and Methods of Making and Using Same,” or are embodied in pending patent applications, such as that found in US20170343466A1 titled “Multiplex Bead Array Assay.” While providing certain advancements in the art of high-speed drug discovery, improvements are still needed in this area.

Accordingly, it is desirable to provide a method in which the interactions of small molecules can be quickly identified and matched to one or more diseases linked with the identified protein. The desired method would provide for the high throughput immobilization of small molecules to a solid matrix followed by rapid identification of relevant interactions.

### SUMMARY OF THE INVENTION

As set forth below, the present inventive concept provides a method of directly conjugating one of a DNA-based barcode, a peptide-based barcode, or a fluorescent microsphere bead-based barcode onto small molecule whereby existing libraries of chemical entities may be modified for better use through the barcoding of small molecules. Barcoded small molecules may be efficiently used in a variety of screening processes. The barcoding process of the present invention utilizes a linker which will effectively allow the linking of any molecule with an aminated molecule. Conventional approaches to linking typically utilize nucleotides. However, according to the methodology of the disclosed concepts, peptides and fluorescent microspheres may be used to improve efficiency. The increased efficiency is partly due to the fact that the peptide and fluorescent microspheres are themselves known to be more stable than many chemicals.

According to the present inventive concept, a barcoded small molecule library is created through the combination of diazirine which is used to attach small molecules to DNA, peptide, and fluorescent microspheres by exposure to ultraviolet radiation. The exposed molecules are then photo-crosslinked resulting in immobilization to the barcodes followed by complementary sequencing. According to the present method, the linker molecule contains a photocoupling group. More particularly, as disclosed, DNA, peptide, or fluorescent microspheres are used which are functionalized with a photo coupling moiety like diazirine directly or through a linker with diazirine on the other end of the linker. This arrangement is used to couple small molecules using UV light exposure as a catalyst to initiate a chemical reaction through the provision of energy.

In each instance a small molecule is provided and is linked using a linker molecule, such as but not absolutely diazirine, which may have a photocoupling agent. On the other side is NH2 or some chemical moiety. The method of preparing the barcode depends on whether the embodiment is DNA-based, peptide-based, or fluorescent bead-based. The amines may be different or may be the same. In the case of peptide-based barcodes and fluorescent microspheres, the amine is automatically provided while for DNA-based barcodes, the amine is attached during synthesis. The NHS reacts with the amine and is immobilized as illustrated. Upon UV activation (~350-360 nm) from 1 minute to 30 minutes, diazirines generate carbene intermediates that rapidly insert into C—H, N—H, O—H, and S—H bonds. The diazirine reacts with multiple areas in the small molecules. In this way the libraries are barcoded. Diazirines are versatile photoreactive molecules that, upon UV irradiation, generate reactive carbenes capable of covalently bonding to a variety of functional groups, such as C—H, N—H, O—H, and S—H bonds. This reactivity enables diazirines to form bonds in an orientation-agnostic manner, meaning they can interact with target molecules without requiring specific spatial alignment. This property makes them particularly valuable for applications like photoaffinity labeling, where they facilitate the study of molecular interactions across diverse orientations and environments.

Immobilization is undertaken preferably through the use of inert material such as barcoded fluorescent magnetic or paramagnetic beads (BMBs) and fluorescent beads composed of a biocompatible polymers are types of beads used in barcoding according to the present invention. The beads may be composed of a variety of materials, including but not limited to polystyrene, a synthetic polymer, and latex The beads are combined resulting in up to 500 uniquely colored beads used to develop barcodes for 500 small molecules simultaneously.

Several small molecules were immobilized using diazirine based linker to microspheres in an orientation agnostic manner. A polypharmacology screening followed. These small molecules included Amikacin, Dexamethasone, Amlodipine, Capsaicin, Nimodipine, Atorvastatin, Sildenafil, Kanamycin, Neomycin, and Losartan. Diazirines generate carbene intermediates that rapidly insert into C—H, N—H, O—H, and S—H bonds present in small molecules. The structures illustrated in FIGS. 1a-1c (Losartan, Nimodipine, and Amikacin [example]) of sample small molecules show that there are many C—H, N—H, O—H bonds in small molecules that can be used to react with carbene intermediates, thus making the crosslinking orientation agnostic, which helps in the screening of these molecules as if a free molecule was being used as a result of the fact that the disclosed method immobilizes each small molecule in multiple orientations on the beads.

With respect to the third embodiment of barcoding, that of fluorescent barcoding using fluorescent micro beads, a fluorophore is tagged to the purified protein of interest or phage comprised of cDNA from any animal tissues used during synthesis of phage display library. Once the fluorophore has been tagged, the purified protein or phage cDNA library with the green fluorophore is introduced to the well plate. The beads are then incubated and washed such that unbound protein or phage are removed from the small molecules by washing. The encoded molecules are then identified as a final step by subjecting the small molecules through the use of an imaging-based analyzer such as the Magpix@ System provided by Luminex@. Other non-limiting systems available for this purpose include the Luminex® 200 and the Luminex Flexmap® 3D.

The present method provides the researcher with a distinct advantage by knowing what molecule is present because of barcoding. Through barcoding, the identity of which molecules are interacting is easily made, thereby saving a significant amount of research time on the part of the technician. The strength of the interactions may be measured by affinity studies using known techniques, such as surface plasmon resonance (SPR).

The disclosed methodology allows for the preparation of a single phage with the specific protein under study. Alternatively, the whole purified protein may be utilized without the use of a phage at all. The disclosed methodology allows for the development of small molecule libraries having barcoding, thereby allowing easy identification of particular molecules of interest to the researcher, a significant advantage if the researcher has a specific target molecule to study or a group of disease targets. The resulting barcoded small molecule phage libraries include different small molecules. In addition, the researcher may study a phage library or can, in the alternative, use the complete transcription.

Other advantages and features of the embodiments of the invention will become apparent when viewed in light of the detailed description of the preferred embodiment when taken in conjunction with the attached drawings and the appended claims.

### BRIEF DESCRIPTION OF THE DRAWINGS

For a more complete understanding of this invention, reference should now be made to the embodiment illustrated in greater detail in the accompanying drawings and described below by way of examples of the invention wherein:

FIG. 1a illustrates Losartan as one sample of a small molecule immobilized using diazirine based linker to microspheres in an orientation agnostic manner according to the present method;

FIG. 1b illustrates Nimodipine as another sample of a small molecule immobilized using diazirine based linker to microspheres in an orientation agnostic manner according to the present method;

FIG. 1c illustrates Amikacin (example) as a further sample of a small molecule immobilized using diazirine based linker to microspheres in an orientation agnostic manner according to the present method;

FIG. 2 is a schematic illustrating the steps involved in barcoding using DNA-based technology according to the present invention;

FIG. 3 is a schematic illustrating the steps involved in barcoding using peptide-based technology according to the present invention;

FIG. 4 is a schematic illustrating the steps involved in barcoding using fluorescent micro bead-based technology according to the present invention;

FIG. 5 is a schematic similar to FIG. 4 but illustrating the tagging of a purified protein with a fluorophore;

FIG. 6 is a schematic illustrating the use of diazirine to attach small molecules to DNA, peptides, or fluorescent microspheres; and

FIG. 7 illustrate diazirine linkers which may be used according to the disclosed inventive method. It is to be understood that while only diazirine linkers are illustrated, other photocoupling agents are available and may be used as linkers according to the present method.

### DETAILED DESCRIPTION OF THE PREFERRED EMBODIMENT

In the following description, various operating parameters and components are described for one constructed embodiment. These specific parameters and components are included as examples and are not meant to be limiting.

The method of the present invention provides a high-throughput screening platform that combines phage display technology or the purified protein or peptide of interest with different approaches to small molecule immobilization. This novel approach has the potential to revolutionize the drug discovery process by enabling rapid and efficient identification of protein-small molecule interactions. The R&D work for this project will focus on three key areas: phage display of proteins, purified proteins, small molecule immobilization on DNA, Peptide or fluorescent microspheres, and assay development and optimization.

More particularly, the present invention provides a tri-modal barcoding system for small molecule libraries using either DNA, peptide, or fluorescent microsphere tags for the screening of libraries of chemical entities which overcomes the problems of high cost and low throughput rate of known methods through the use of tri-modal barcoding and linkers. Particularly, the present method provides a high-throughput screening platform that incorporates barcoding small molecules for use in screening whole premade, synthesized, small molecule libraries of chemical entities through the use of DNA-based, peptide-based, and fluorescent micro bead-based barcoding. This technology is combined with phage display for rapid identification of small molecule-protein interactions. This method of the present invention addresses several key challenges in drug discovery and represents a significant advancement in screening methodologies. One aspect of the core innovation lies in the integration of microsphere technology such as provided by Luminex® (Luminex Corporation), utilizing fifty-five hundred distinct fluorescent microsphere sets each coupled to a different small molecule of interest, and phage display, employing phage particles displaying the proteins or purified proteins of interest labeled with green fluorophores.

The combination of technologies allows for multiplexed screening, enabling the simultaneous testing of multiple small molecules against a protein target or a group of proteins in a single well. It also significantly increases throughput, allowing for the screening of thousands of interactions in a fraction of the time required by traditional methods. Furthermore, the miniaturization of the assay reduces sample consumption, requiring minimal amounts of valuable proteins and compounds.

The disclosed barcoding method builds upon multiplexing technology such as that made available from Luminex® xMAP® (Luminex Corporation) technology, which has been used for multiplexed immunoassays and nucleic acid detection, and phage display techniques, traditionally used for antibody discovery and protein engineering. By combining these technologies, Applicant created a platform that leverages the strengths of both approaches while overcoming their individual limitations.

The disclosed barcoding method optimizes the phage display system to efficiently express a diverse library of proteins on the surface of bacteriophages. This involves designing and constructing phage vectors or protein expression vectors for protein expression, developing protocols for efficient protein display or expression while maintaining proper folding and functionality, and establishing quality control measures to ensure consistent protein display or expression across the library.

Additionally, the disclosed barcoding method provides a robust method to couple small molecules to fluorescent microspheres. This includes optimizing coupling chemistry for diverse small molecule structures, ensuring uniform distribution and density of small molecules on microsphere surfaces, and verifying the stability and accessibility of immobilized small molecules. Applicant found that the resulting barcodes demonstrated a high degree of stability under all of the experimental conditions, including during exposure to ultraviolet radiation as well as washing.

Furthermore, the disclosed barcoding method involves integrating the phage display and microsphere components into a functional screening assay. This includes developing protocols for efficient green fluorophore tagging of phage particles or purified proteins, optimizing incubation conditions to promote specific binding while minimizing non-specific interactions, establishing washing procedures to effectively remove unbound phage, and calibrating an imaging-based analyzer such as the Luminex® MagPix® instrument for sensitive and accurate detection of binding events. This work proves technical feasibility by demonstrating that proteins can be efficiently displayed on phage while retaining their binding properties, small molecules can be successfully immobilized on microspheres without losing their ability to interact with target proteins, and the integrated system can detect specific protein-small molecule interactions with high sensitivity and specificity.

The method of the present invention significantly reduces technical risk by addressing several key challenges. By optimizing the phage display system, a high percentage of phage particles display functional proteins is ensured, increasing the likelihood of identifying rare but valuable interactions. The immobilization techniques of the present invention ensures that small molecules remain accessible for protein binding, reducing the risk of false negatives due to steric hindrance. The resulting optimization of the fluorophore tagging and detection protocols improve the signal-to-noise ratio, enabling the identification of weak but potentially important interactions. Demonstrating the compatibility of this approach with high-throughput screening instruments addresses concerns about the scalability of the technology.

The method of the present invention significantly accelerates the identification of lead compounds and protein targets, potentially reducing the time and cost of early-stage drug development through faster screening and compatibility with diverse libraries. The commercial viability of this platform is supported by the growing demand for more efficient drug discovery tools in the pharmaceutical industry, the potential for this technology to address challenging targets that are difficult to approach with traditional methods, and the compatibility with existing high throughput screening infrastructure, facilitating adoption by research laboratories and pharmaceutical companies. In addition, the method of the present invention may find application in a variety of other uses, such as in chemical biology research or diagnostics. The method involves novel combinations of existing technologies to create an innovative screening platform, addressing significant technical challenges in protein display, small molecule immobilization, and assay development, and exploring the fundamental interactions between displayed proteins and immobilized small molecules in a high-throughput format. The method requires the integration of multiple complex biological and chemical systems resulting in a transformative drug discovery tool.

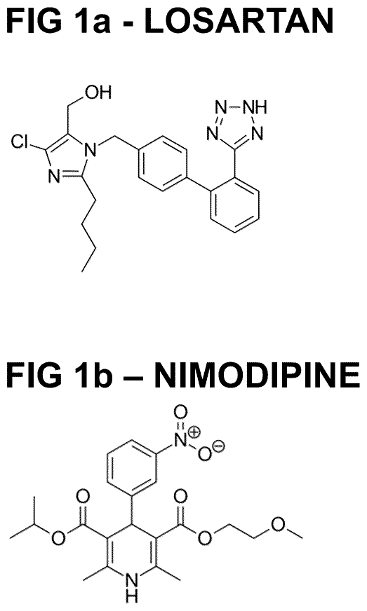

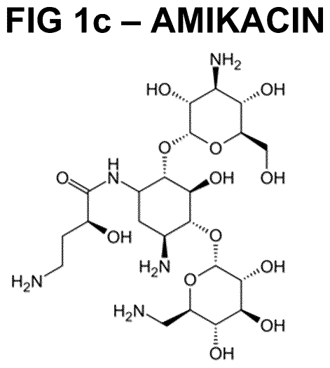

Referring to FIG. 1, a schematic illustrating the steps involved in barcoding using DNA-based technology according to the present invention is shown. This technique involves the creation of a DNA-encoded library (“DEL”). DELs are conventionally created through the use of “split-and-pool” combinatorial chemistry. According to this strategy, a precursor is created and is then split into wells according to conventional techniques. The number of wells used in this strategy varies although the conventional well arrangement includes 96 wells. The reactions are pooled followed by re-splitting. This chemistry-tagging process may be repeated several times. The resulting DEL-encoded library comprises DNA molecular barcodes. According to this technique, a starting material or scaffold such as a DNA fragment is selected. To this starting material is added a new building block (BB1, BB2, etc.) followed by the step of adding a unique DNA barcode to the building block. An amine is attached during synthesis. The steps are repeated with each step adding different chemical modifications and barcodes. The resulting pool of barcoded DNA is then utilized in creating a chemical compound through a linking with the barcoded DNA.

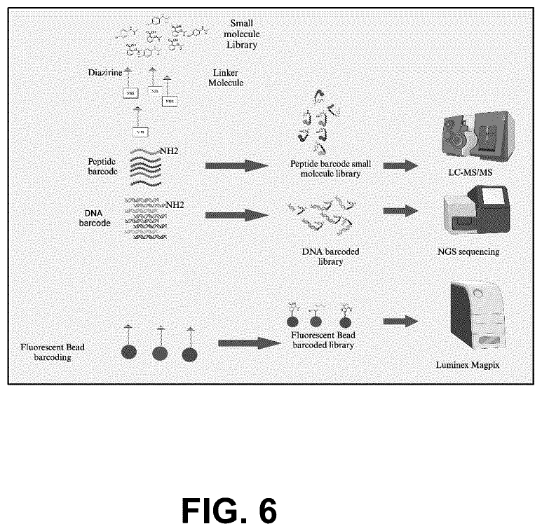

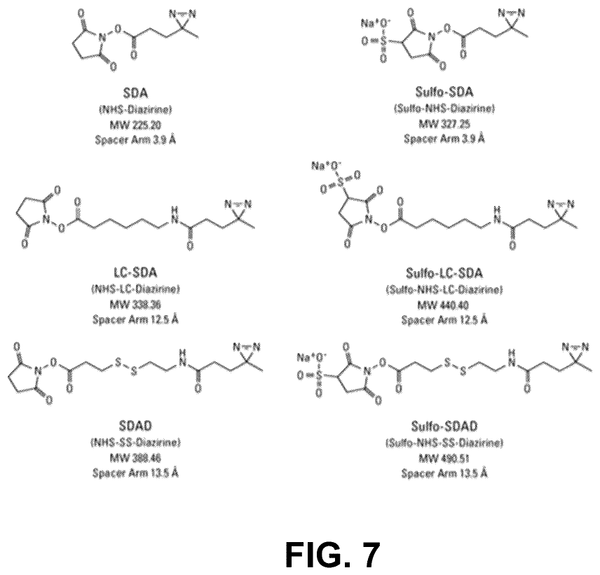

At the first step of the method of FIG. 1, a selected diazirine (included with the DNA) or as a linker is combined with a small molecule library. Linkage of the diazirine-linked DNA may be achieved through the use of a variety of linkers, such as a bifunctional linker and other possible linkers illustrated in FIG. 6 which has diazirine moiety on one end where small molecules are immobilized and an N-hydroxysuccinimide (NHS) moiety on the other end used for linking the DNA barcode. (The barcoded library will be used in biopanning with phage display library of peptides and proteins. Biopanning is a known technique for affinity selection which selects for peptides binding to a given target.)

According to the present invention, photo-coupling reactions utilize various chemical moieties to enable light-induced coupling of different molecules. These photoreactive groups play a crucial role in facilitating bond formation upon light activation. The following are examples of chemical moieties used for photo-coupling according to the disclosed method.

### Azide-Based Moieties

Aryl Azides: These compounds, such as 4-azido-N-ethyl-1,8-naphthalimide (AzNP), are highly stable in aqueous solutions and can be converted to reactive aryl nitrene species upon light activation. AzNP in particular emits strong green fluorescence, shows good photo-crosslinking ability, and can covalently attach to proteins upon UV illumination. Other azide-based moieties include phenyl azide, benzoyl azide, and vinyl azide. However, some of these are less stable in physiological systems.

### Carbonyl-Based Moieties

Phenacyl Groups: These aryl ketones undergo photoinitiated homolytic scission of bonds, making them useful as photoremovable protecting groups (PPGs) for various molecules. Key features include of phenacyl groups of carbonyl-based moieties include their absorption in the near-UV region and their ability to release molecules like enzymes, neurotransmitters, and signaling molecules.

### Diazo and Diazirine Moieties

These groups can be converted to reactive carbene species upon UV light activation. They are characterized by having a short lifetime (<2 ns) in aqueous solution and upon UV activation (350-360 nm) from 1 minute to 30 minutes, diazirines generate carbene intermediates that rapidly insert into C—H, N—H, O—H, and S—H bonds present in small molecules.

### Photoredox Catalysts

While not moieties themselves, these catalysts enable photo-induced coupling reactions. These catalysts facilitate electron transfer processes crucial for coupling reactions. They include iridium-based complexes like and acridinium-based photocatalysts.

### Halogen-Containing Groups

lodoalkynes: These compounds can be directly activated by visible light, acting as “alkynyl radical synthetic equivalents” for coupling with various C(sp2)—H bonds.

### Semiconductor-Based Materials.

Graphitic carbon nitride (g-C3N4) based materials serve as photocatalysts for various coupling reactions, including hydrogen evolution, water oxidation, CO2 reduction, and organic transformations.

By utilizing these diverse chemical moieties and photocatalysts, researchers can achieve a wide range of photo-coupling reactions, enabling the synthesis of complex molecules and materials under mild, light-driven conditions.

At the next step following linkage, each molecule is exposed individually (having a separate bar code) to ultraviolet radiation, preferably long wave radiation (such as, but not limited to, 360 nm). The photo cross-linking of small molecules is then undertaken resulting in immobilization to DNA molecular barcodes.

The resulting barcoded library and a phage displayed or purified protein or fluorescent beads are mixed and incubated. At this step, the protein and the barcoded library are incubated together with the protein or phage displaying protein. After thorough washing the molecules binding to the protein are identified by sequencing the peptide through LC-MS/MS or the DNA encoded molecules are identified by through PCR and sequencing of DNA while the fluorescent microspheres can be identified using instruments such as Luminex® Magpix®.

To be suitable for the synthesis of DNA-encoded chemical libraries (DEL), a reaction must be compatible with the DNA to reduce the loss of the genetic information to a minimum (Malone and Paegel, 2016). Strongly acidic reaction conditions, oxidants, and many transition-metal ions compromise the integrity of DNA oligonucleotides, for example, by depurination. Extreme reaction conditions such as prolonged reaction at very high temperatures is not compatible with DNA. In addition, to be suitable for the DEL synthesis, any reaction must fulfill further requirements. As DNA is insoluble in many organic solvents the reaction should tolerate water or aqueous solvent mixtures. The reactions have to provide defined products, high yields, should show a broad reactant scope, and include low by-product formation to ensure the homogeneous representation of library members (Franzini and Randolph, 2016).

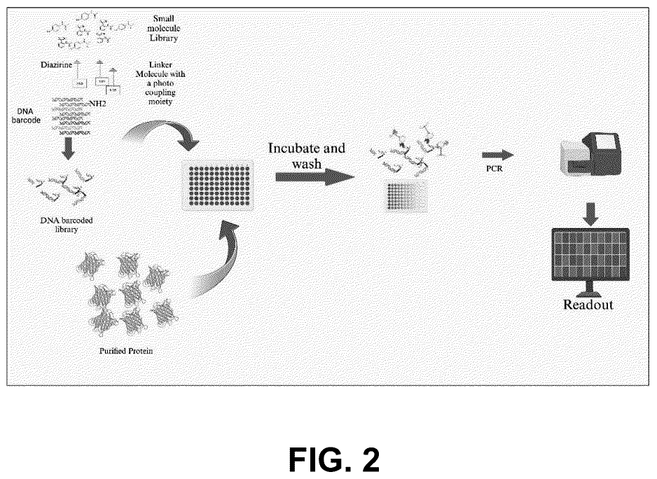

Referring to FIG. 2, a schematic illustrating the steps involved in the creation of a peptide encoded library according to the method of the present invention is illustrated.

Peptide encoded libraries result from the use of peptides as carriers of information for the barcoding of small-molecule library synthesis. Such libraries can be created using a number of techniques, including combinatorial synthesis, truncation, randomization, epitope display, and split intein circular ligation. In addition, peptide encoded libraries can also be created by a split-and-pool synthesis, an approach that is generally taken to create a large number of compounds. According to this technique, a unique peptide sequence having an amine attached is selected for attachment to a preferred small molecule. The technique involves functionalizing different peptides with a photocoupling moiety like diazirine with or without a linker to initially to couple a specific peptide with a specific small molecule and create an encoded mixture of compounds (the library) to bind to a particular protein. Thereafter, during the decoding process, the mixture is subjected to washing to remove proteins or phage that do not bind. Mass spectrometry may then be used to decode the peptides which are attached to the molecules that are bound to a protein of interest (the protein target). Molecules can then be created without the peptide tag.

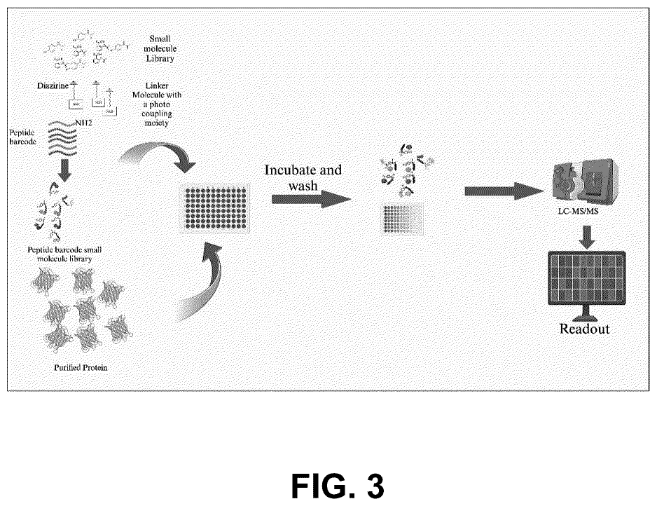

Referring to FIG. 3, a schematic illustrating the steps involved in the creation of a small molecule fluorescent bead-based library using fluorescent micro bead-based technology. According to the illustrated method, an imaging-based analyzer is used to measure several analytes simultaneously in a single microplate well. According to this system, the beads are immobilized by use of a magnetic force. This same force excites the beads by way of a light source, such as light-emitting diodes. The relevant beads and analytes are then detected using an optical device such as a CCD camera. An example of such a system is the MagPix® made available by Luminex®.

An alternative method of analyzing the results of fluorescent bead-based assays relies upon the use of flow cytometry. According to this method, the fluorescent beads are excited using lasers. The results are analyzed through the use of real-time, digital processing to differentiate both bead color (the analyte) and the signal strength resulting from fluorescence intensity of protein or phage tagged with a green fluorophore. Software for use according to this method is available from Luminex® under the brand name xPONENT®.

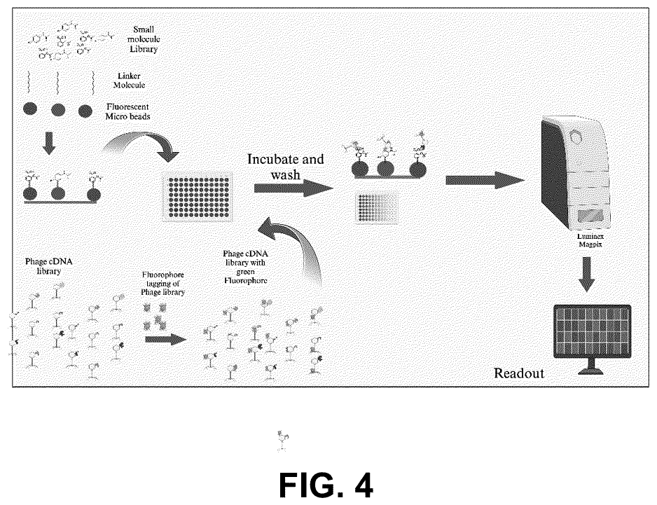

Referring to FIG. 4, a method illustrating the tagging of a purified protein with a fluorophore is shown. The illustrated method of FIG. 4 is similar to that of FIG. 3 but differs from FIG. 3 insofar as in FIG. 3 a fluorophore is tagged to a phage that displays a protein of interest. Particularly, as shown in FIG. 4, the method provides for the use of fluorescent microspheres in an assay with either purified protein tagged with green fluorophore or phage library tagged with green fluorophore according to the present invention is illustrated.

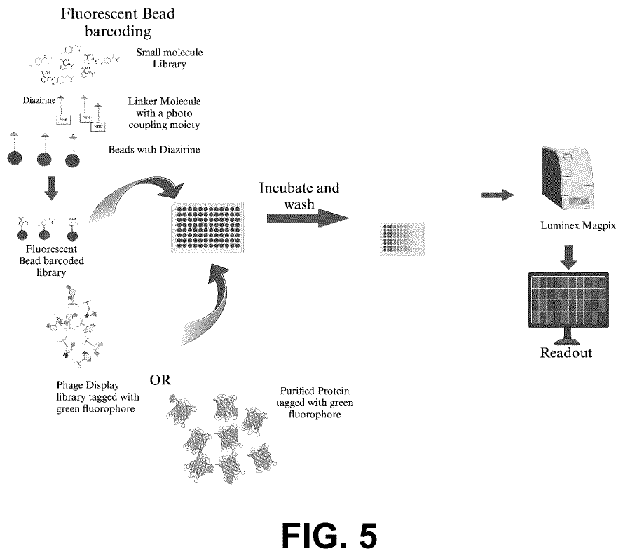

Referring to FIG. 5, the illustrated methods set forth the use of diazirine to attach small molecules to DNA, Peptide and fluorescent microspheres. The diazirine moiety can be functionalized to the DNA, peptide or fluorescent microspheres during their synthesis or can be attached to them using a linker with diazirine on one side and other moiety like NHS to attach to any one of an amine group, a carboxyl group, or a similar group. The DNA, peptide, or fluorescent microspheres can be directly functionalized with a diazirine moiety. For this reason, the fluorescent microspheres are placed separately in the figures with diazirine as well as the linker.

With respect to FIG. 6, non-limiting examples of linkers for use in the present invention are illustrated. It is to be understood the illustrated linkers are only meant to be suggestive as it is possible that other linkers may effectively be employed for use in the present method.

### Case Study—Biotinylation of T7 Phage

Phage PEG precipitation

T7 phage was added to day cultures of BW 25113 (OD~0.7) at an MOI of ~0.001, and then placed into a shaking incubator (250 RPMs, 37° C.) for 2 h. After 2 h, the cultures were removed, and chloroform was added to a concentration of 0.5% (v/v) and then vigorously shaken. This mixture was left for an hour at room temperature. The phage lysates were then placed into 50 mL falcon tubes and centrifuged at 4000 rpms for 20 min at room temperature. Following centrifugation, a separation of chloroform and lysate emerged, and the phage lysate was removed (top layer) and filtered using sterile syringe filters (0.2 um polyether sulfone (PES) membrane from VWR®). Thereafter, 5 g of PEG (MW: 8000) and 4.87 g. NaCl was added to each 50 mL tube of phage lysate. This solution was then shaken until all of the solid was dissolved and incubated overnight at 5° C. After incubation, the solution was spun down at 12.1 k rpm in an Eppendorf fixed angle 50 mL rotor for an hour. A phage pellet sedimented at the bottom of the tubes and the excess solution was discarded to waste. The pellets were then resuspended in 500 μL of PBS (1×).

### Ultracentrifugation Purification

Cesium Chloride was the medium used for ultracentrifugation. For making layered density gradients, 0.7 g/mL, 0.9 g/mL, and 1.1 g/mL CsCl solutions were prepared in AAS buffer (0.1 M. ammonium acetate, 10 mM NaCl, 1 mM CaCl₂, 5 mM MgCl₂, pH 7) 26. SYBR Safe (10,000×) was also added to improve phage visualization. A SW41Ti rotor was used (Beckman) for these experiments with Ultra-Clear™ Tubes (Beckman 344059). The volume of these tubes is 13.2 mL so 3.3 mL of each density gradient was added layering from the lightest gradient to the heaviest from the bottom of the tube with 1 mL of phage PEG precipitate added to the top. The centrifuge was run at 25,000 rpm (100,000×g) for 2 h at 8° C. to establish equilibrium. Once completed, the tubes were visualized and imaged using an LED transilluminator (Invitrogen dual-led blue-white LED) and a smart phone camera, respectively. Samples were collected by puncturing the tube with a needle and drawing up the phage. ImageJ processing program was used for image analysis. The CsCl was then removed from the phage sample via a 48-h. dialysis cycle in 1×PBS in a dialysis membrane with a molecular weight cut off of 14,000 (Sigma). Plaquing assays were carried out to determine the efficiency of the purification process.

### Phage Biotinylation

The S-NHS-Biotin (EZ-Link™ NHS-Biotin, Catalog No. 20217) was dissolved in DMSO at a concentration of 34 mM (in 1 mL of DMSO). After cesium chloride (CsCl) density gradient ultracentrifugation and dialysis, 1.83×10¹¹ PFUs of T7 was mixed with S-NHS-Biotin (APExBIO), volumetrically as 1/50 of total volume, 1 M NaH₂PO₄ (pH 7), producing a total volume of 136.3 μL. This was then reacted for 12 h in the dark at room temperature. The resulting biotinylated-phage lysate was then dialyzed to remove excess biotin. Throughout this process at every step of functionalization and purification the phage activity was examined through plaquing for each step to determine activity.

### Biolayer Interferometry (BLI) Assay

BLI studies were performed using an Octet ® N1 instrument (Sartorius) at 23° C. with shaking at 2200 RPM (or 37° C. for formula studies). Prior to using Amine Reactive 2nd Generation (AR 2G) or Streptavidin (SA) biosensors, they were hydrated in autoclaved nanopure water (18.2 Mohm) for 10 min. Samples and buffer steps were conducted in black 0.6 mL tubes with each tube having a volume of 400 μL. The steps of the BLI method were run in a series of these tubes. Tube 1 was PBS-T (0.1%). Tubes 2-5 were a series of biotinylated phage samples ranging from 2×10⁵ to 3×10⁸ PFU/mL. The series was prepared through 1:10 sequential dilutions from the most concentrated sample, 3×10⁸ PFU/mL, in 1×PBS. Tubes 3-6 were PBS-T (0.1%) for wash steps. Tube 7 was BW25113 or BW25113ΔwaaCΔtrxA or ECOR strain suspended in PBS after four washing cycles. The bacterial samples were a 5 mL overnight culture (OD ~2.0) concentrated to 400 μL. Tubes 8-11 was PBS-T (0.1%) for wash steps. Tube 12 was a 400 μL aliquot of Luria-Bertani (LB) broth from Difco™.

The BLI method was run as follows. Tube 1 (equilibration) for 300 s, Tube 2 (loading) for 3600 s, Tube 3 (wash 1) for 300 s, Tube 4 (wash 2) for 300 s, Tube 5 wash 3) for 300 s, Tube 6 (wash 4) for 300 s, Tube 7 (bacterial addition) for 3600 s, Tube 8 (wash 5) for 300 s, Tube 9 (wash 6) for 300 s, Tube 10 (wash 7) for 300 s, Tube 11 (wash 8) for 300 s, Tube 12 (LB incubation) for 30,000 s (overnight). Results were recorded in Octet® BLI software via the sensor gram. The raw data was then exported as a .csv file for graphing and interpretation of specific steps.

Common photo-activatable groups used in the study include aryl azides, benzophenones, diazirines, and most recently, 2-aryl-5-carboxytetrazoles (ACTs).

The present invention provides several advantages over known technologies. Through the integration of microsphere technology and phage display, small molecule-protein interaction screening results in efficient and high-speed screening techniques, having the potential for revolutionizing drug discovery by accelerating the identification of lead compounds, enabling the discovery of novel protein-ligand interactions, and reducing costs and time associated with early-stage drug development. The broad applicability of the disclosed platform across various fields, including drug discovery, chemical biology, and proteomics, further emphasizes its potential high impact. By addressing these challenges, the project aims to create a transformative tool for drug discovery and protein science, potentially leading to faster and more efficient development of new therapeutics.

The foregoing discussion discloses and describes an exemplary embodiment of the present invention. One skilled in the art will readily recognize from such discussion, and from the accompanying drawings and claims that various changes, modifications and variations can be made therein without departing from the true spirit and fair scope of the invention.

I claim:

1. A method for creating a barcoded small molecule phage library comprising the steps of:
selecting a small molecule library;
conjugating a barcode onto the small molecule of the library using a tri-modal barcoding system by combining the selected small molecules of the small molecule library with an organic or inorganic molecular tags or carrier molecule through the use of a linker molecule;
exposing the combined molecules to radiation;
immobilizing the combined and irradiated molecules on inert material;
washing the molecules; and
identifying the remaining protein-bound molecules.

2. The method of claim 1, wherein the linker molecule contains a photocoupling group.

3. The method of claim 1, wherein the linker molecule is diazirine for radiation-induced coupling.

4. The method of claim 1, wherein the linker molecule is a bifunctional linker.

5. The method of claim 1, wherein the radiation is ultraviolet radiation.

6. The method of claim 5, wherein the ultraviolet radiation is in the range of 350-360 nm.

7. The method of claim 1, wherein the radiation is long wave radiation.

8. The method of claim 1, wherein the inert material is a matrix.

9. The method of claim 8, wherein the inert material is an affinity matrix.

10. The method of claim 1, wherein the inert material are barcoded fluorescent beads.

11. The method of claim 10, wherein the fluorescent beads are selected from the group consisting of magnetic, paramagnetic, polystyrene, synthetic, and latex beads.

12. The method of claim 1, wherein the inert material comprises a biocompatible polymer.

13. The method of claim 1, including subjecting the washed molecules to PCR.

14. The method of claim 1, including the step of identifying the remaining protein-bound molecules is accomplished using an imaging-based analyzer.

15. The method of claim 1, in which the step of immobilization is repeated.

16. The method of claim 1, wherein the barcode is a DNA-based barcode.

17. The method of claim 16, including the step of attaching an amine during synthesis.

18. The method of claim 1, wherein the barcode is a peptide-based barcode.

19. The method of claim 18, wherein an amine is attached.

20. The method of claim 1, wherein the barcode is a fluorescent microsphere bead-based barcode.

21. The method of claim 20, wherein an amine is attached.

22. The method of claim 1, further including integration of microsphere technology, the use of microsphere sets each coupled to a different small molecule, and phage display, employing phage particles displaying the proteins or purified proteins of interest labeled with green fluorophores.
